(19) United States
(12) Patent Application Publication
(10) Pub. No.: US 2008/0306133 A1
(43) Pub. Date: Dec. 11, 2008

(54) INTRANASAL ADMINISTRATION OF ASENAPINE AND PHARMACEUTICAL COMPOSITIONS THEREFOR

(76) Inventors: Josephine Elisabeth Maria van der Sterren, Wijchen (NL); Dennie J.M. van den Heuvel, Beuningen (NL)

Correspondence Address: SYNTHON IP INC, 7130 HERITAGE VILLAGE PLAZA, STE 202, GAINESVILLE, VA, 20155, US

(21) Appl. No.: 12/133,106

(22) Filed: Jun. 4, 2008

Related U.S. Application Data
(60) Provisional application No. 60/942,082, filed on Jun. 5, 2007.

(51) Int. Cl. A61K 31/407; A61P 25/18

(52) U.S. Cl. 514/412

(57) ABSTRACT

Asenapine or a pharmaceutically acceptable salt thereof can be administered intranasally, typically via an intranasal dosage formulation having a water-containing liquid carrier.

This application claims the benefit of priority under 35 U.S.C. § 119(e) from prior U.S. provisional application Ser. No. 60/942,082, filed Jun. 5, 2007, the entire contents of which are incorporated herein by reference.

### BACKGROUND OF THE INVENTION

1. Field of the Invention

The present invention relates to the intranasal administration of asenapine and to dosage formulations useful for such administration.

2. Description of the Prior Art

Many pharmaceutical compounds that exert their activity in the brain, such as antipsychotics, are administered orally. During oral delivery, the drug passes down the digestive tract and is absorbed into blood capillaries of the duodenum, jejunum, and ileum, enters the portal vein, and is then transported to the liver before reaching the target organ, the brain. Unfortunately, oral delivery of such compounds can sometimes be disadvantageous due to, inter alia, inferior uptake or absorption in the GI tract and/or high hepatic first pass metabolism, which results in low overall bioavailability.

Asenapine, which is the generic name for trans-5-chloro-2-methyl-2,3,3a,12b-tetrahydro-1H-dibenz[2,3:6,7]oxepino[4,5-c]pyrrole of the formula (1)

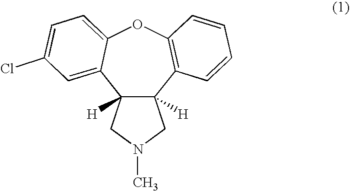

is a compound having CNS-depressant activity (see Boer et al., Drugs of the Future, 18(12), 1117-1123, 1993) and is completing Phase III clinical trials for the treatment of schizophrenia and bipolar disorder. Asenapine was reported as being effective at a dose of 10 mg/day (5 mg twice daily) in the treatment of schizophrenia, as assessed by the Positive and Negative Symptoms Scale (PANSS) in randomised, double-blind, placebo-controlled trials. The compound and pharmaceutically acceptable salts thereof were disclosed in U.S. Pat. No. 4,145,434.

The bioavailability of an oral dosage form of asenapine, however, is apparently very low (<1%), which may be caused by an extensive first pass metabolism in the gastrointestinal tract or in the liver.

WO 95/23600 (EPB 746317, U.S. Pat. No. 5,763,476) report that oral doses of asenapine may have cardiovascular side effects and propose the use of sublingual and/or buccal dosage forms of asenapine to lessen these side effects. The sublingual and/or buccal pharmaceutical compositions are preferably solid compositions that rapidly disintegrate in the mouth, although a simple form of the composition is described as an aqueous solution containing 0.9% (w/v) sodium chloride and asenapine. The maleate salt of asenapine is taught to be the preferred asenapine compound. The physico-chemical properties of the asenapine maleate had been previously reported by Funke et al. (Arzneim.-Forsch/Drug Res., 40, 536-539, 1990).

The buccal cavity, including the sublingual and buccal tissues, often affords slow drug absorption. Moreover drugs delivered in the mouth can be bitter and/or irritating. It would be desirable to administer asenapine via an alternative route.

### SUMMARY OF THE INVENTION

The present invention is based on the idea of administering asenapine or its salts via a nasal route. Accordingly, a first aspect of the invention relates to an intranasal dosage formulation, which comprises asenapine or a pharmaceutically acceptable salt thereof and a water-containing liquid carrier, wherein the formulation is adapted for intranasal administration. Often the liquid carrier also comprises a polyol, e.g., an alkylene glycol or polyalkylene glycol such as propylene glycol or polyethylene glycol. The formulation is adapted for intranasal administration such that administering the asenapine active agent via this route is practical. A permeation enhancing agent, which can be the polyalkylene glycol or another type as described below, is typically present in the formulation to increase or aid the uptake of the asenapine active via the nasal mucosa.

Another aspect of the invention relates to a method which comprises administering via intranasal exposure an effective amount of asenapine or a pharmaceutically acceptable salt thereof to a patient in need thereof. The patient is typically suffering from an antihistamine or antiserotonin related condition such that he or she is in need of asenapine or its pharmaceutically acceptable salt. A particular condition to be treated is schizophrenia by delivering an anti-schizophrenia effective amount of asenapine or its salt via nasal administration. The intranasal administration of asenapine is conveniently carried out using the above intranasal dosage formulation.

### DETAILED DESCRIPTION OF THE INVENTION

The present invention relates to the intranasal administration of asenapine or its pharmaceutically acceptable salt (for convenience the base and salts of asenapine are frequently referred to herein after as simply “asenapine.”). An intranasal exposure to asenapine allows for transport into the central nervous system (CNS) of a human or other mammal by the nasal mucosa. In general, the exposure allows or facilitates paracellular transport through the nasal mucosa. “Paracellular transport” refers to the transfer of substances between (as opposed to transcellular transport through) epithelial cells of the nasal mucosa. The nasal epithelium has good permeability and a good blood supply. Asenapine can be well absorbed through the nasal mucosa, thereby avoiding any first-pass metabolic effect that may occur after oral administration. The rich vascular plexus of the nasal mucosa of a mammal provides a direct route into the bloodstream for compounds that readily cross mucous membranes, such as asenapine. Due to direct absorption into the bloodstream, problems of gastrointestinal destruction and hepatic first-pass metabolism are avoided, thereby improving the net bioavailability of the asenapine relative to oral delivery.

The intranasal exposure or administration of the asenapine should minimize the transport of asenapine from the nasal passages into the lungs. That is, the intranasal dosing of the present invention is not an inhalation route of administration, but as explained above, is an intranasal route. The dosage formulation and/or equipment used to administer the asenapine active agent should serve to restrict inhalation and contact of the formulation with the nasal turbinates and oropharynx. Suitable dosage equipment includes containers with droppers as well as gas aerosol sprayers. The droplet size of the formulation as administered should generally be larger than about 20 μm up to 100 μm or larger, so that the administered droplets immediately drop to the nasal mucosa and do not enter the lungs. While a few droplets potentially can escape and enter the oropharynx and subsequently be swallowed, essentially no material will enter the lungs in the form of an aerosol.

Asenapine can be administered via the intranasal method of the present invention for any therapeutic purpose(s) for which asenapine is useful/suitable; hereinafter “an asenapine-treatable condition.” Generally, asenapine is used to treat antihistamine and antiserotonin related conditions or diseases in a patient. Examples of asenapine-treatable conditions include tension, excitation, anxiety, and psychotic and schizophrenic disorders. The intranasal method is expected to be useful in treating mental illness and disorders in a convenient way. From a therapeutic standpoint, the intranasal administration of asenapine may provide improved duration, more efficient and precise control, and/or a more rapid onset of activity as compared to conventional methods.

Asenapine or its pharmaceutically acceptable salt is typically administered in the form of an intranasal dosage formulation. The formulation normally contains asenapine or a pharmaceutically acceptable salt thereof and a water-containing liquid carrier and is adapted for intranasal administration. A formulation that is “adapted for intranasal administration” permits a sufficient tolerability and drug permeation vis-a-vis the nasal mucosa, and preferably has similar properties with respect to nasal secretions (e.g., similar pH, viscosity, and/or ionic strength), so that normal ciliary action is maintained. The water-containing liquid carrier contains water alone or in combination with other water miscible solvents such as alcohol(s). In many embodiments the liquid carrier advantageously contains water and a polyol. A polyol includes polyalkylene glycols such as polyethylene glycol (e.g. PEG 3000), as well as simpler polyols such as propylene glycol. The polyols can serve to increase the solubility of the asenapine, e.g. asenapine maleate, in the liquid carrier and/or to improve the stability of the formulation. The amount of the polyol relative to the water is generally within the range of 10:90 to 90:10 (v/v), but more typically about 20:80 to about 80:20. In some embodiments the amount of water and polyol(s) is generally equal, that is 50:50+/−10, while in other embodiments the polyol is at least 50% of the liquid carrier such as 60 to 85% of the carrier (by volume). The amount of polyol depends in part on the intended concentration of the asenapine as well as other factors including overall formulation viscosity, etc. For example, a high molecular weight polyol may, at a certain concentration in a particular formulation, provide sufficient solubility but result in the formulation being too sticky for convenient administration. Using less of such a high molecular weight polyol may resolve the stickiness concerns but could reduce the stability or practical asenapine concentration. The high molecular weight polyol could be mixed with other lower molecular weight polyols and/or alcohols, e.g. ethanol, to obtain the desired balance of asenapine concentration and stickiness or viscosity.

The formulation is typically a solution, although certain ingredients including the asenapine may be suspended therein. Emulsions and gel or gel-like suspensions are also contemplated for the intranasal dosage form of the present invention.

The dosage formulation can be a single dose or multiple doses. When administered, the dose volume of the formulation is typically less than 0.9 ml per nostril, generally 0.2 ml or less per nostril, and often preferably less than or equal to 0.1 ml per nostril. Because asenapine (and pharmaceutically acceptable salts thereof) is effective at dosages of about 10 mg/daily within a twice a day administration, the concentration of the asenapine (and pharmaceutically acceptable salts thereof) in the formulation is typically in the range of 5 to 100 mg/ml. As an example, at a concentration of 25 mg of asenapine per 1 ml of the intranasal dosage formulation, the formulation can be administered at a daily dose of 10 mg of asenapine to a patient by a twice daily administration of 100 μl of the formulation in each nostril. It should be noted, however, that a higher net bioavailability that is achievable by the intranasal administration of the present invention may allow for lower dosing levels of the asenapine active to achieve the same therapeutic plasma levels. In such a case, the concentration of the asenapine or its salt may be lower (including 0.5 to 10 or 20 mg/ml), and/or smaller dosage volumes may be used. On the other hand, small dosing volumes are preferred for intranasal administration in order to minimize unintentional swallowing, and thus higher concentrations are also preferred, e.g., at least 10 mg/ml, generally at least 20 mg/ml and typically within the range of about 20 to about 200 mg/ml. The upper end of the concentration is generally determined by the solubility of the asenapine in the liquid carrier/formulation and the practical volume to be administered. Often the asenapine concentration is within the range of 20 to about 100 mg/ml and includes values of about 25, about 40, about 50, about 60, and about 80 mg/ml.

Water soluble salts of asenapine are generally preferred salts within the present invention. For purposes of the present invention, a water soluble salt of asenapine has a solubility of at least 5 mg/ml in water at a pH of about 4.0 to about 5.0. Asenapine maleate is an example of such a salt, exhibiting a solubility in water of about 10 mg/ml at a pH of about 4.0 to about 5.0 (i.e., 1 mg/100 μl). If desired, a non-water soluble salt or asenapine free base, can be formulated at enhanced concentrations by incorporating a suitable solubilizer (e.g., benzyl alcohol) into the dosage formulation or the more general use of a polyol as discussed above.

The pH of the intranasal dosage formulation is generally in a range of 4.0 to 8.0, preferably in a range of 4.0 to 6.0. A buffering agent may be used to adjust the overall pH of the formulation to be within this range. Any pharmaceutically acceptable buffering agent may be used, e.g. a citrate buffer. If the asenapine is present as a salt, then the pH of the composition may be adjusted/buffered by adding an aqueous base or acid, e.g. NaOH or HCl.

One or more permeation enhancing agents are generally used in the intranasal dosage formulation to obtain a desired absorption rate of the asenapine or pharmaceutically acceptable salt thereof. In this respect, the nature and/or amount of the agent(s) is not specifically restricted.

Suitable permeation-enhancement agents include, alone or in a combination, an absorption enhancing agent or system, an aggregation inhibitory agent, a degradative enzyme inhibitory agent, a mucolytic or mucus clearing agent, a ciliostatic agent, a modulatory agent of epithelial junction physiology, a vasodilator agent, and a complex-forming species. Liquid permeation-enhancement agents may also be considered as liquid carriers; e.g., forming part of the water-containing liquid carrier of the formulation.

The absorption enhancing agent includes (i) a surfactant; (ii) a bile salt (including sodium taurocholate); (iii) a phospholipid additive, mixed micelle, or liposome; (iv) an alcohol (including a polyol as discussed above, for example, propylene glycol or polyethylene glycol such as PEG 3000, etc.); (v) an enamine; (vi) a nitric oxide donor compound; (vii) a long-chain amphipathic molecule; (viii) a small hydrophobic uptake enhancer; (ix) sodium or a salicylic acid derivative; (x) a glycerol ester of acetoacetic acid; (xi) a cyclodextrin or cyclodextrin derivative; (xii) a medium-chain or short-chain (e.g. C1 to C12) fatty acid; and (xiii) a chelating agent; (xiv) an amino acid or salt thereof; and (xv) an N-acetylamino acid or salt thereof.

The aggregation inhibitory agent may be selected from, e.g., surfactants, NaCl, KCl, and sugars (including poloxamers). The degradative enzyme inhibitory agent may be selected from, e.g., amastatin, sodium glycocholate, and a trypsin inhibitor. The mucolytic or mucus clearing agent may be selected from, e.g., methionine, cysteine, and threonine. The ciliostatic agent may be selected from, e.g., benzalkonium chloride, EDTA, and bile salts. The modulatory agent of epithelial junction physiology may be selected from, e.g., EDTA, citric acid, and salicylates. The vasodilator agent may be selected from, e.g., arginine and polyarginine. The complex-forming species may be selected from, e.g., cyclodextrins and EDTA.

Typically, the total concentration of the one or more permeation enhancing agent(s) of the formulation is 0.1 to 800 mg/ml, generally not more than 250 mg/ml. Moreover, the ratio of the asenapine or pharmaceutically acceptable salt thereof to the one or more permeation enhancing units is not particularly limited and can range from about 50:1 to about 1:8. For example, the ratio may be about 50:1, 41:1, 21:1, 9:1, 8:1, 1:2, 1:5, or 1:8.

In addition, the formulations of the present invention may further comprise one or more additives, such as a bacteriostatic (antiseptic) agent, a liquid solubility enhancer, an isotonizing agent, a thickener, and a humectant (anti-irritant).

Bacteriostatic agents are useful for repeated administrations of the formulation, which often places the formulation in contact with environmental air so that aerial bacteria may contaminate the formulation and, as a result, the nasal cavity. The bacteriostatic agents minimize the danger of such contamination. Useful bacteriostatic agents include, e.g., benzalkonium chloride and EDTA, as they also improve the absorption of the drug through the nasal mucosa.

Solubility enhancers may increase the concentration of the asenapine or pharmaceutically acceptable salt thereof in the formulation. Useful solubility enhancers include, e.g., alcohols and polyalcohols.

An isotonizing agent may improve the tolerance of the formulation in a nasal cavity. A common isotonizing agent is NaCl. Preferably, when the formulation is an isotonic intranasal dosage formulation, it includes about 0.9% NaCl (v/v) in the aqueous portion of the liquid carrier.

The thickeners may improve the overall viscosity of the composition, preferably to values close to those of the nasal mucosa. Suitable thickeners include methylcellulose, carboxymethylcellulose, polyvinypyrrolidone, sodium alginate, hydroxypropylmethylcellulose, and chitosan.

A humectant or anti-irritant improves the tolerability of the composition in repeated applications. Suitable compounds include, e.g. glycerol, tocopherol, mineral oils, and chitosan.

The intranasal dosage formulations of the present invention may be prepared by any suitable means. In general the components are combined by dissolving weighed amounts of the asenapine or pharmaceutically acceptable salt thereof and the optional additional components (if desired) in the liquid carrier, or a portion thereof, in one or more steps. For example, when a solubility enhancer such as a polyol is present, the asenapine may be dissolved therein and then the water component added to dilute the formulation, though such is not required. Regardless of how combined, the resulting solution may optionally be filtered through a 0.2μ filter. The pH can then be recorded and adjusted with a NaOH or HCl solution, if necessary. Instead of using purified water, the formulations may be prepared with a NaCl solution (e.g., an about 0.9% NaCl solution) to obtain an isotonic intranasal dosage formulation.

The compositions of the present invention are suitable for pharmaceutical applications. They may be used for intranasally administering a pharmaceutically effective amount of asenapine or a salt thereof to patients in need thereof; e.g., patients suffering from an asenapine-treatable condition. In particular, a patient in need of asenapine typically suffers from an antihistamine or antiserotonin treatable condition. A particular condition to be treated is schizophrenia by delivering an anti-schizophrenia effective amount of asenapine or its salt via nasal administration; likewise for treating bipolar disorder.

The invention will be further described with reference to the following non-limiting examples.

### EXAMPLES

| Composition | Amount (g) |
|---|---|
| Asenapine maleate | 2.5 |
| PEG 3000 | 20 |
| 0.9% NaCl | q.s. to 40 ml |
| 0.1 M NaOH ad pH = 5 | |
| Asenapine maleate | 2.5 |
| PEG 3000 | 20 |
| Carboxymethylcellulose | 5 |
| 0.9% NaCl | q.s. to 40 ml |
| 0.1 M NaOH ad pH = 5 | |
| Asenapine maleate | 1.0026 |
| PEG 3000 | 8.00 |
| 0.9% NaCl | q.s. to 40 ml |
| 0.1 M NaOH ad pH = 5 | |
| Asenapine maleate | 1.0024 |
| PEG 3000 | 8.00 |
| Carboxymethylcellulose | 2.00 |
| 0.9% NaCl | q.s to 40 ml |
| 0.1 M NaOH ad pH = 5 | |
| Asenapine maleate | 0.2493 |
| Propylene glycol | 8.0 |
| 0.9% NaCl | q.s. to 10 ml |
| 0.1 M NaOH ad pH = 5 | |
| Asenapine maleate | 2.010 |
| Propylene glycol | 8.1 |
| 0.9% NaCl | q.s. to 10 ml |
| benzalkonium Cl* | 0.0065 |
| 0.1 M NaOH ad pH = 5 | |
| Asenapine maleate | 1.004 |
| Propylene glycol | 4.1 |
| Benzalkonium Cl* | 0.0063 |
| 0.9% NaCl | q.s. to 10 ml |
| 0.1 M NaOH ad pH = 5 | |
| Asenapine maleate | 0.998 |
| Propylene glycol | 5.0 |
| Benzalkonium Cl* | 0.0039 |
| 0.9% NaCl | q.s. to 10 ml |
| 0.1 M NaOH ad pH = 5 | |

*A 50% Benzalkonium chloride solution was prepared in water

The excipients, except as noted hereinafter, were weighed and added to a glass vial. The asenapine maleate was added. The vials were then subsequently filled with NaCl solution to the desired level (e.g. 40 or 10 ml). The pH was adjusted with NaOH solution under stirring.

Each of the patents, patent applications and journal articles mentioned above is incorporated herein by reference. The invention having been described it will be obvious that the same may be varied in many ways and all such modifications are contemplated as being within the scope of the invention as defined by the following claims.

We claim:

1. An intranasal dosage formulation, which comprises:
asenapine or a pharmaceutically acceptable salt thereof and a water-containing liquid carrier, wherein the formulation is adapted for intranasal administration.

2. The dosage formulation according to claim 1, wherein said water-containing liquid carrier comprises water and a polyol.

3. The dosage formulation according to claim 2, wherein said polyol is polyethylene glycol or propylene glycol.

4. The dosage formulation according to claim 3, wherein said water and said polyol are present in a volume ratio within the range of 20:80 to 80:20.

5. The dosage formulation according to claim 1, wherein said formulation contains asenapine or a pharmaceutically acceptable salt thereof in a concentration within the range of 5 to 100 mg/ml.

6. The dosage formulation according to claim 1, which further comprises a bacteriostatic agent.

7. The dosage formulation according to claim 1, which further comprises a buffering agent.

8. The dosage formulation according to claim 1, which further comprises a permeation enhancing agent selected from the group consisting of:
(a) an absorption enhancing agent;
(b) an aggregation inhibitory agent;
(c) a degradative enzyme inhibitory agent;
(d) a mucolytic or mucus clearing agent;
(e) a ciliostatic agent;
(f) a modulatory agent of epithelial junction physiology;
(g) a vasodilator agent; and
(h) a complex-forming species.

9. The dosage formulation according to claim 8, wherein said permeation enhancing agent is selected from the group consisting of a surfactant, a bile salt, a phospholipid additive, a mixed micelle, a liposome, an alcohol, an enamine, a nitric oxide donor compound, a salicylic acid derivative, a glycerol ester of acetoacetic acid, a cyclodextrin, a cyclodextrin derivative, a C1-C12 fatty acid, an amino acid or salt thereof, a complexing agent, a trypsin inhibitor, a sugar, and combinations thereof.

10. The dosage formulation according to claim 8, wherein said permeation enhancing agent is selected from the group consisting of an N-acetylamino acid or salt thereof, NaCl, KCl, amastatin, sodium glycocholate, methionine, cysteine, threonine, benzalkonium chloride, EDTA, citric acid, a poloxamer, a polyol, a salicylate, arginine, polyarginine, and combinations thereof.

11. The dosage formulation according to claim 1, wherein the formulation has a pH in a range of 4.0 to 6.0.

12. The intranasal dosage formulation of claim 1, wherein the pharmaceutically acceptable salt of asenapine is asenapine maleate.

13. An intranasal dosage formulation, which comprises:
asenapine or a pharmaceutically acceptable salt thereof in a concentration of 10 to 100 mg/ml;
a liquid carrier comprising water and a polyol;
an isotonizing agent;
optionally a bacteriostatic agent; and
optionally a thickener and/or a humectant.

14. The dosage formulation according to claim 13, wherein said asenapine or pharmaceutically acceptable salt thereof is asenapine maleate.

15. The dosage formulation according to claim 14 wherein the polyol is polyethylene glycol, propylene glycol, or combinations thereof.

16. The dosage formulation according to claim 15, wherein said asenapine maleate is contained in an amount within the range of 20 to 100 mg/ml.

17. A method, which comprises administering via intranasal exposure an effective amount of asenapine or a pharmaceutically acceptable salt thereof to a patient in need thereof.

18. The method according to claim 18, wherein said patient is suffering from schizophrenia and said amount of asenapine is an anti-schizophrenia effective amount.

19. A method for treating asenapine-treatable conditions, which comprises intranasally administering an effective amount of the intranasal dosage formulation according to claim 1, to a patient suffering from an asenapine-treatable condition.

20. The method according to claim 19, wherein said patient is suffering from schizophrenia and said amount administered is an anti-schizophrenia effective amount of asenapine.
